(19) United States
(12) Patent Application Publication
(10) Pub. No.: US 2024/0090790 A1
(43) Pub. Date: Mar. 21, 2024

(54) SYSTEM AND METHOD FOR IMAGING ORGANS WITH MULTIPLE BLOOD SUPPLIES

(71) Applicant: Wisconsin Alumni Research Foundation, Madison, WI (US)

(72) Inventor: Mark Kliewer, Madison, WI (US)

(21) Appl. No.: 17/946,481

(22) Filed: Sep. 16, 2022

(51) Int. Cl. A61B 5/055; G01R 33/28; G01R 33/56

(52) U.S. Cl. CPC A61B 5/055; G01R 33/281; G01R 33/5601; A61B 2503/02

(57) ABSTRACT

A system and method are provided for generating images of a portion of a subject including an organ receiving blood from multiple, different supplies. The method includes accessing MR data acquired from a subject having received a dose of a material configured to change MR contrast. The method also includes reconstructing the MR dataset into a first set of MR images using a first reconstruction process configured to weight the first set of MR images to the material configured to change the MR contrast and reconstructing the MR dataset into a second set of MR images using a second reconstruction process configured to weight the second set of MR images against the material configured to change the MR contrast. The method further includes displaying the first set of MR images and the second set of MR images to selectively display blood flow from each of the multiple, different supplies.

### BACKGROUND

The present disclosure relates to systems and methods for imaging organs with multiple blood supplies. More specifically, the present disclosure provides systems and methods for distinguishing each of the blood supplies to a given organ in an efficient and consistent manner that meets clinical requirements.

Some organs receive blood from multiple, distinct supplies, such as the liver. In such cases, it may be clinically valuable to be able to separately view the dynamics of each blood supply separately, for example, to assess the flow and function of each and better understand how a given supply operates to serve the organ. Unfortunately, it is often difficult, impractical, inefficient, or even impossible to fully investigate each blood supply separately using non-interventional techniques, such as medical imaging.

Thus, it would be desirable to have systems and methods for distinguishing each of multiple blood supplies to a given organ in an efficient and consistent manner that meets clinical requirements.

### SUMMARY OF THE DISCLOSURE

The present disclosure provides systems and methods for imaging an organ, for example, the placenta, that receives blood flow from multiple different supplies, where each of the different supplies are independently distinguishable in the reconstructed images. In accordance with one non-limiting example, a magnetic resonance imaging (MRI) system may be used to acquire an MRI dataset from the multiple different supplies simultaneously, which creates an efficient acquisition process and eliminates the need to register images acquired across multiple different acquisitions. During reconstruction of the MRI dataset, a first reconstruction is performed that yields images of a first of the multiple different supplies and a second reconstruction is performed that yields images of the second of the multiple different supplies. The images of the first and the second of the multiple different supplies can be displayed together or separately.

In accordance with one aspect of the disclosure, a magnetic resonance (MR) imaging system is provided that includes a magnet system configured to generate a static magnetic field (BO) about at least a portion of a subject including an organ receiving blood from multiple, different supplies and having received a dose of a material configured to change MR contrast and a plurality of gradient coils configured to apply magnetic gradients to the static magnetic field. The MR system also includes a radio frequency (RF) system configured to apply an excitation field to the subject and acquire MR image data from the subject and a computer system. The computer system is programmed to acquire an MR dataset from the organ receiving the blood from multiple, different supplies, reconstruct the MR dataset into a first set of MR images using a first reconstruction process configured to weight the first set of MR images to the material configured to change the MR contrast, and reconstruct the MR dataset into a second set of MR images using a second reconstruction process configured to weight the second set of MR images against the material configured to change the MR contrast. The MR system also includes a display configured to display the first set of MR images and the second set of MR images together with a user-selectable weighting of the first set of MR images relative the second set of MR images to independently view the multiple, different supplies of blood to the organ.

In accordance with another aspect of the disclosure, a method is provided for imaging a portion of a subject including an organ receiving blood from multiple, different supplies using a magnetic resonance imaging (MRI) system. The method includes delivering a dose of a material configured to change MR contrast to the subject and, following delivery of the dose of the material configured to change MR contrast to the subject, simultaneously acquiring MR data from multiple, different supplies to form an MR dataset. The method also includes reconstructing the MR dataset into a first set of MR images using a first reconstruction process configured to weight the first set of MR images to the material configured to change the MR contrast and reconstructing the MR dataset into a second set of MR images using a second reconstruction process configured to weight the second set of MR images against the material configured to change the MR contrast. The method further includes receiving a user-selectable weighting of the first set of MR images relative the second set of MR images to independently view the multiple, different supplies of blood to the organ and displaying the first set of MR images and the second set of MR images together based on the user-selectable weighting of the first set of MR images relative the second set of MR images to independently view the multiple, different supplies of blood to the organ.

In accordance with yet another aspect of the disclosure, a computer system is provided including a non-transitory computer-readable medium having stored thereon instructions that, when executed by the computer, cause the computer to carry out steps. The steps include accessing MR data acquired from a region of interest (ROI) including a placenta of a subject having received a dose of a material configured to change MR contrast. The steps also include reconstructing the MR data into a set of maximum intensity projection (MIP) images and reconstructing the MR data into a set of minimum intensity projection (MinIP) images. The steps also include receiving a user selection of one of a transparency or a weighting of the MIP images relative to the MinIP images and displaying the MIP images and the MinIP images as a composite image of the placenta formed based on the user selection.

In accordance with still another aspect of the disclosure, a method is provided for creating images of a placenta of a subject pregnant with a fetus. The method includes accessing MR data acquired from a region of interest (ROI) including a placenta of a subject having received a dose of a material configured to change MR contrast and reconstructing the MR dataset into a first set of MR images using a first reconstruction process configured to weight the first set of MR images to placental blood flow of the subject. The method also includes reconstructing the MR dataset into a second set of MR images using a second reconstruction process configured to weight the second set of MR images to placental blood flow of the fetus and displaying the first set of MR images and the second set of MR images together to selectively display placental blood flow of the subject or placental blood flow of the fetus

In accordance with another aspect of the disclosure, a method is provided for generating images of a portion of a subject including an organ receiving blood from multiple, different supplies. The method includes accessing MR data acquired from a subject having received a dose of a material configured to change MR contrast, wherein the MR data was acquired from a region of interest (ROI) including an organ with blood flow from multiple, different supplies. The method also includes reconstructing the MR dataset into a first set of MR images using a first reconstruction process configured to weight the first set of MR images to the material configured to change the MR contrast and reconstructing the MR dataset into a second set of MR images using a second reconstruction process configured to weight the second set of MR images against the material configured to change the MR contrast. The method further includes displaying the first set of MR images and the second set of MR images to selectively display blood flow from each of the multiple, different supplies.

These and other advantages and features of the invention will become more apparent from the following detailed description of the preferred embodiments of the invention when viewed in conjunction with the accompanying drawings.

### DESCRIPTION OF THE DRAWINGS

FIG. 1 is a block diagram of an exemplary magnetic resonance imaging (MRI) system configured in accordance with the present disclosure.

FIG. 2 is a flow chart setting forth some non-limiting examples of steps of a process in accordance with the present disclosure.

FIG. 3 is a non-limiting set of example images of one visualization of a report produced by the system and methods of the present disclosure.

### DETAILED DESCRIPTION

Placental function is critical for the health and well-being of both mother and fetus. To date, imaging of placental anatomy has been markedly limited in detail. The limitations of current imaging systems and methods persist despite an ongoing and unmet need to images the placenta to assist with diagnosis and treatment of important conditions, such as placenta accreta spectrum and placental insufficiency leading to fetal growth restriction (FGR or IUGR).

Placenta accreta spectrum is a potentially life-threatening pregnancy complication that occurs in approximately 1 in 1000 to 2000 pregnancies. It occurs when the placenta grows too deeply into the wall of the uterus and is unable to detach at childbirth.

Fetal growth restriction is defined as a pathologic decrease in the rate of fetal growth. The most frequent etiology for late onset fetal growth restriction is uteroplacental dysfunction which is due to inadequate supply of nutrients and oxygen to support normal aerobic growth of the fetus. However, fetal chromosomal anomalies, structural anomalies and fetal infections should be carefully excluded. The condition can lead to problems both for mother and fetus. It is linked to pre-eclampsia and placental abruption, when the placenta peels away from the wall of the uterus. There can be serious complications for the fetus, including lack of oxygen during the birth, premature labor, low blood sugar, too little calcium in the blood, or too many red blood cells, and sometimes even stillbirth.

When FGR is suspected or detected, ultrasound imaging is commonly ordered and, in some cases, additional MR imaging is used to try and determine if there are structural abnormalities. Unfortunately, often neither imaging modality is able to provide images suitable for diagnosis or desired to sufficiently guide treatment. With proper diagnosis, different treatments would be prescribed. For example, if there is a thrombosis, blood thinners would be prescribed. On the other hand, if a fibrin issue is detected, early delivery would be suggested.

Unfortunately, to properly diagnose and develop a clinical course of action, it is generally necessary to distinguish maternal blood flow from fetal blood flow. As with most organs that receive blood from multiple, distinct supplies, including the liver or ovaries, this is clinically impractical or impossible with traditional imaging systems and protocols.

For example, using MRI, one option for distinguishing just one blood supply to an organ receiving blood from multiple supplies includes performing arterial spin labeling. In this case, one must “tag” the upstream spins and then image the excited or saturated spins entering the organ. In this case, each supply must be tagged separately and requires the ability to carefully tag only the spins in the vessels heading into the organ. This means that multiple acquisitions must be performed (one for each supply) and the images must be registered. Furthermore, the tagging must be very precise, else the contrast expected for the supply vessels will be obscured by spins flowing elsewhere. These limitations and others make ASL generally impractical for such clinical applications.

As another example, one may attempt to deliver a contrast agent, such as gadolinium, into a one supply. This can be impractical for some organs because precise access to the only one blood supply at a time is unavailable. That is, a generally arterial dose of contrast agent would result in an imprecise diffusion of contrast through the vascular system, resulting in substantial portions of the vascular system reflecting similar contrast at the same time, thereby obscuring the ability to distinguish one supply from the other.

For example, angiographic studies using magnetic resonance imaging (MRI) or computed tomography (CT) imaging rely on contrast agents to enhance the signal from the vascular system. In an organ such as the liver, complex studies have been devised where contrast is delivered arterially to only enhance the hepatic artery before contrast reaches the veins and then enters the liver through the portal vein too. This requires careful administration of the contrast agent and, it is often impractical or impossible to deliver the contrast to only one blood supply at a time.

Other imaging modalities are also unsuitable. For example, computed tomography presents similar challenges with contrast management and necessitates the patient receiving a dose or doses of ionizing radiation. Ultrasound, while avoiding ionizing radiation, has a limited field of view and presents the same challenges with managing contrast in a way that enables one to differentiate blood supplies.

Thus, clinicians have not found traditional imaging modalities and protocols practical for separately visualizing or otherwise understanding multiple supplies of blood delivered to a given organ. Thus, clinical protocols have been developed that do not rely on imaging, for example, when diagnosing FGR. Unfortunately, however, the result can be improper diagnosis or delayed diagnosis, which can result in negative clinical outcomes.

The present disclosure overcome these and other drawbacks by providing systems and methods for imaging an organ, for example, the placenta, that receives blood flow from multiple different supplies, where each of the different supplies are independently distinguishable in the reconstructed images. In accordance with one non-limiting example, a magnetic resonance imaging (MRI) system may be used to acquire an MRI dataset from the multiple different supplies simultaneously, which creates an efficient acquisition process and eliminates the need to register images acquired across multiple different acquisitions. During reconstruction of the MRI dataset, a first reconstruction is performed that yields images of a first of the multiple different supplies and a second reconstruction is performed that yields images of the second of the multiple different supplies. The images of the first and the second of the multiple different supplies can be displayed together or separately.

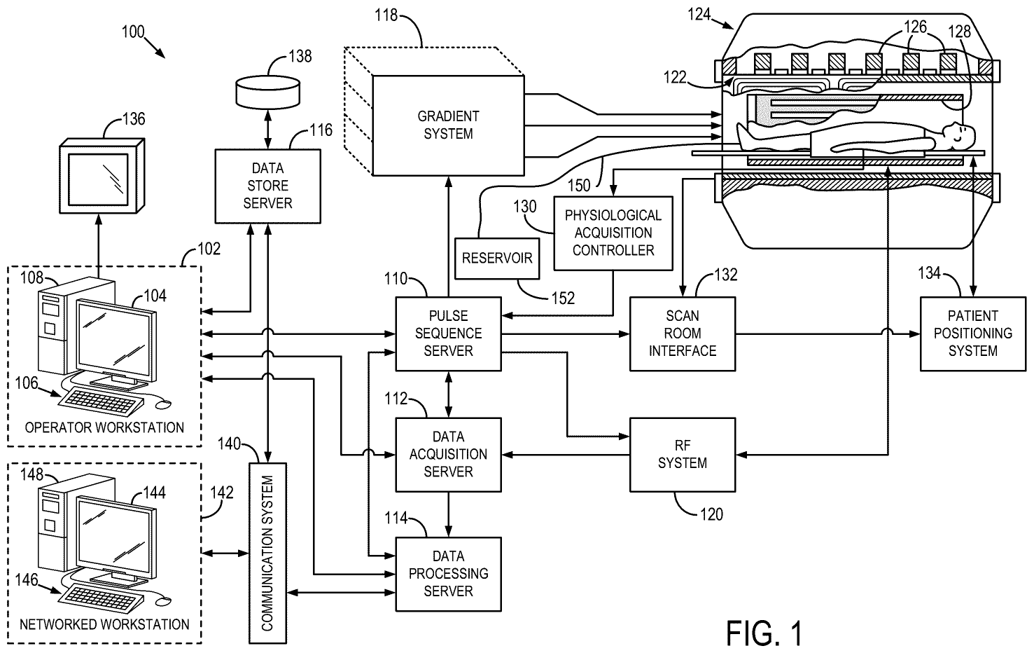

For example, referring now to FIG. 1, a magnetic resonance imaging (MRI) system 100 is provided that may be configured, programmed, or otherwise utilized in accordance with the present disclosure. The MRI system 100 includes an operator workstation 102, which will typically include a display 104, one or more input devices 106 (such as a keyboard and mouse or the like), and a processor 108. The processor 108 may include a commercially available programmable machine running a commercially available operating system. The operator workstation 102 provides the operator interface that enables scan prescriptions to be entered into the MRI system 100. In general, the operator workstation 102 may be coupled to multiple servers, including a pulse sequence server 110; a data acquisition server 112; a data processing server 114; and a data store server 116. The operator workstation 102 and each server 110, 112, 114, and 116 are connected to communicate with each other. For example, the servers 110, 112, 114, and 116 may be connected via a communication system 140, which may include any suitable network connection, whether wired, wireless, or a combination of both. As an example, the communication system 140 may include both proprietary or dedicated networks, as well as open networks, such as the internet.

The pulse sequence server 110 functions in response to instructions downloaded from the operator workstation 102 to operate a gradient system 118 and a radiofrequency (RF) system 120. Gradient waveforms to perform the prescribed scan are produced and applied to the gradient system 118, which excites gradient coils in an assembly 122 to produce the magnetic field gradients Gₓ, G_{y}, G_{z} used for position encoding magnetic resonance signals. The gradient coil assembly 122 forms part of a magnet assembly 124 that includes a polarizing magnet 126 and a whole-body RF coil 128.

RF waveforms are applied by the RF system 120 to the RF coil 128, or a separate local coil (not shown in FIG. 1), in order to perform the prescribed magnetic resonance pulse sequence. Responsive magnetic resonance signals detected by the RF coil 128, or a separate local coil, are received by the RF system 120, where they are amplified, demodulated, filtered, and digitized under direction of commands produced by the pulse sequence server 110. The RF system 120 includes an RF transmitter for producing a wide variety of RF pulses used in MRI pulse sequences. The RF transmitter is responsive to the scan prescription and direction from the pulse sequence server 110 to produce RF pulses of the desired frequency, phase, and pulse amplitude waveform. The generated RF pulses may be applied to the whole-body RF coil 128 or to one or more local coils or coil arrays.

The RF system 120 also includes one or more RF receiver channels. Each RF receiver channel includes an RF preamplifier that amplifies the magnetic resonance signal received by the coil 128 to which it is connected, and a detector that detects and digitizes the I and Q quadrature components of the received magnetic resonance signal. The magnitude of the received magnetic resonance signal may, therefore, be determined at any sampled point by the square root of the sum of the squares of the I and Q components:

M=\sqrt{I²+Q²} Eqn. 1;
and the phase of the received magnetic resonance signal may also be determined according to the following relationship:

$$φ = {tan}^{- 1} ( \frac{Q}{I} ) . \qquad Eqn . 2$$

The pulse sequence server 110 also optionally receives patient data from a physiological acquisition controller 130. By way of example, the physiological acquisition controller 130 may receive signals from a number of different sensors connected to the patient, such as electrocardiograph (ECG) signals from electrodes, or respiratory signals from a respiratory bellows or other respiratory monitoring device. Such signals are typically used by the pulse sequence server 110 to synchronize, or “gate,” the performance of the scan with the subject's heartbeat or respiration.

The pulse sequence server 110 also connects to a scan room interface circuit 132 that receives signals from various sensors associated with the condition of the patient and the magnet system. It is also through the scan room interface circuit 132 that a patient positioning system 134 receives commands to move the patient to desired positions during the scan.

The digitized magnetic resonance signal samples produced by the RF system 120 are received by the data acquisition server 112. The data acquisition server 112 operates in response to instructions downloaded from the operator workstation 102 to receive the real-time magnetic resonance data and provide buffer storage, such that no data are lost by data overrun. In some scans, the data acquisition server 112 does little more than pass the acquired magnetic resonance data to the data processor server 114. However, in scans that require information derived from acquired magnetic resonance data to control the further performance of the scan, the data acquisition server 112 is programmed to produce such information and convey it to the pulse sequence server 110. For example, during prescans, magnetic resonance data are acquired and used to calibrate the pulse sequence performed by the pulse sequence server 110. As another example, navigator signals may be acquired and used to adjust the operating parameters of the RF system 120 or the gradient system 118, or to control the view order in which k-space is sampled.

The data processing server 114 receives magnetic resonance data from the data acquisition server 112 and processes it in accordance with instructions downloaded from the operator workstation 102. Such processing may, for example, include one or more of the following: reconstructing two-dimensional or three-dimensional images by performing a Fourier transformation of raw k-space data; performing other image reconstruction techniques, such as iterative or backprojection reconstruction techniques; applying filters to raw k-space data or to reconstructed images; generating functional magnetic resonance images; calculating motion or flow images; and so on.

Images reconstructed by the data processing server 114 are conveyed back to the operator workstation 102. Images may be output to operator display 112 or a display 136 that is located near the magnet assembly 124 for use by attending clinician. Batch mode images or selected real time images are stored in a host database on disc storage 138. When such images have been reconstructed and transferred to storage, the data processing server 114 notifies the data store server 116 on the operator workstation 102. The operator workstation 102 may be used by an operator to archive the images, produce films, or send the images via a network to other facilities.

The MRI system 100 may also include one or more networked workstations 142. By way of example, a networked workstation 142 may include a display 144, one or more input devices 146 (such as a keyboard and mouse or the like), and a processor 148. The networked workstation 142 may be located within the same facility as the operator workstation 102, or in a different facility, such as a different healthcare institution or clinic. The networked workstation 142 may include a mobile device, including phones or tablets. The networked work station 142 may function as a picture archiving and communication system (PACS) terminal.

The networked workstation 142, whether within the same facility or in a different facility as the operator workstation 102, may gain remote access to the data processing server 114 or data store server 116 via the communication system 140. Accordingly, multiple networked workstations 142 may have access to the data processing server 114 and the data store server 116. In this manner, magnetic resonance data, reconstructed images, or other data may be exchanged between the data processing server 114 or the data store server 116 and the networked workstations 142, such that the data or images may be processed remotely by a networked workstation 142. This data may be exchanged in any suitable format, such as in accordance with the transmission control protocol (TCP), the internet protocol (IP), or other known or suitable protocols.

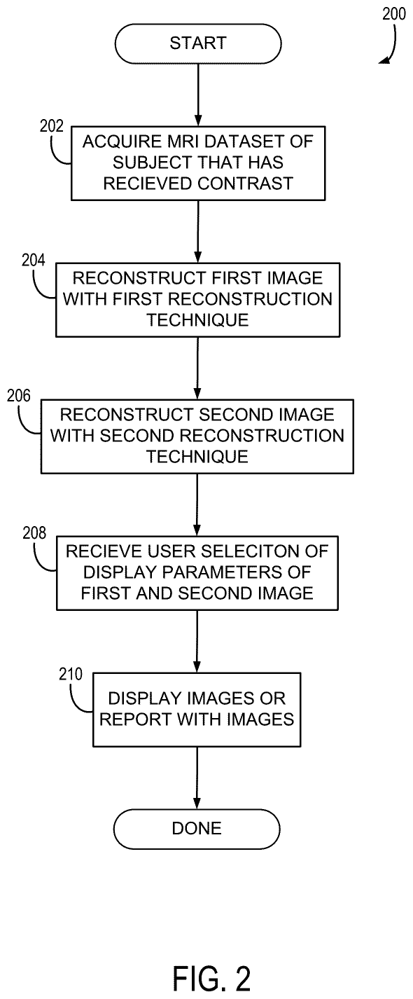

The above-described system can be utilized in accordance with the present disclosure to image an organ that receives blood flow from multiple different supplies, where each of the different supplies are independently distinguishable in the reconstructed images. Referring to FIG. 2, a flow chart of one, non-limiting method 200 starts by acquiring an MRI dataset of a region of interest (ROI) in a subject that has received a dose of contrast at process block 202. The ROI includes the organ that receives blood flow from multiple different supplies. That is, the subject has received a contrast agent designed to enhance MR contrast, but is to be delivered to only one of the multiple different supplies. The “acquiring” may include using an MRI system 100 such as described above with respect to FIG. 1, or “acquiring” may include accessing the MRI dataset from storage or memory. Thus, the process may be performed on an MRI workstation or a remote computer or other workstation. For example, the methods described herein may be performed with previously-acquired MRI datasets, for example, via a PACS terminal or other computer or mobile computing system, or a full MRI system may be utilized to acquire a new MRI dataset from a patient.

In one non-limiting example, the organ is the placenta and the contrast agent may be or include Ferumoxytol. Ferumoxytol is a benign therapeutic intravenous drug utilized to treat maternal iron-deficiency anemia during pregnancy. However, in accordance with the present disclosure, Ferumoxytol may be used as an MRI contrast agent. Furthermore the Ferumoxytol may be delivered only to the material circulatory system, such that only blood supplied to the placenta from the mother will include the Ferumoxytol. The present disclosure recognizes that Ferumoxytol does not cross the placenta and, thus, a dose of Ferumoxytol as an MR contrast agent can be administered to the mother without concern that the contrast will enhance contrast of the blood traveling to the placenta from the fetus. Thus, at process block 202, the MR dataset is acquired from an ROI including the placenta and the acquisition need not be specifically timed to avoid enhancement of the blood traveling to the placenta from the fetus. In fact, only one MR acquisition is required that acquires a single dataset from the organ (e.g., placenta) and multiple different supplies of blood to the organ.

At process block 204, a first set of images is reconstructed from the MR dataset using a first reconstruction technique. In accordance with one, non-limiting example, the first reconstruction technique may be a maximum intensity projection reconstruction. A maximum Intensity Projection (MIP) projects the pixel or voxel with the highest attenuation value on every view throughout the volume onto an image. Thus, the first reconstruction technique yields a first image of the ROI that includes the organ (e.g., placenta) and multiple different supplies of blood to the organ. As described above, the contrast agent will have enhanced blood supplied to the organ from the one supply that received the contrast agent. In the non-limiting example above, the material blood flow may be enhanced and, thus, will dominate the MIP image.

At process block 206, a second set of images is reconstructed from the same MR dataset using a second reconstruction technique that is different from the first reconstruction technique. In accordance with one, non-limiting example, the second reconstruction technique may be a minimum intensity projection reconstruction. A minimum Intensity Projection (MinIP) projects the pixel or voxel with the lowest attenuation value on every view throughout the volume onto an image. Thus, the second reconstruction technique yields a second image of the ROI that includes the organ (e.g., placenta) and multiple different supplies of blood to the organ. As described above, the contrast agent will have enhanced blood supplied to the organ from the one supply that received the contrast agent. In the non-limiting example above, the material blood flow may be enhanced and, thus, will not be included in the MinTP image.

At process block 208, a user may communicate selected parameters for displaying the first and second images. For example, the first and second images, because they were reconstructed from the same or common MR dataset, are perfectly registered. Thus, the user may select to display the images with one stacked on top of the other. In this way, the parameters may indicate whether the first image or the second image should be on top and/or may indicate a transparency or opacity of the first or second image. Furthermore, the parameters may indicate a color coding or graphic visualization that distinguishes the two images and, thereby, the two distinct blood supplies.

At process block 210, the parameters communicated by the user are used to display the images or a report that may include the images. In accordance with the non-limiting example described above, the images or report may depict the intraplacental anatomic structures and the maternal and fetal circulations, whereby the Ferumoxytol-enhanced supplies are readily distinguished from the non-enhanced supplies. Depiction of this anatomy in vivo is heretofore unprecedented and, thus, provides clinicians with new insights into placental function during pregnancy and allows therapeutic interventions with new clarity and understanding.

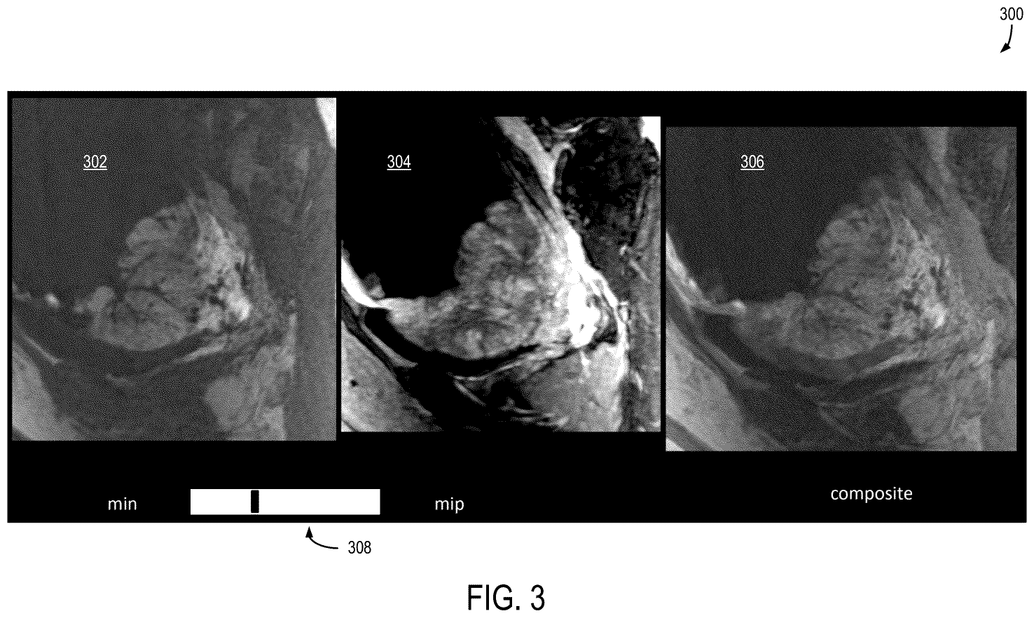

In particular, referring now to FIG. 3, one non-limiting example of a selected display or report 300 is illustrated. In the non-limiting example, three images may be displayed along side each other. For example, a first, MinIP or min image 302; a second, MIP image 304; and a third, composite image 306 may be displayed or included in a report. A user interface 308 may be included that allows a user to control how the composite image 306 is displayed. For example, in the non-limiting example illustrated in FIG. 3, the user interface 308 may include a slider that allows the user to select between a greater or lesser weighting of the MIN image 302 or the MIP image 304. Additionally or alternatively, the user interface 308 may control an opacity or transparency of the MIN image 302 relative to the MIP image 304 or vice versa. Furthermore, the report can include findings on these images that can be correlated with pathologic diagnoses, post-delivery, to establish correlative features of various placental diseases, and further improve diagnosis in the future.

As such a system and method is provided to depict, for example, the intraplacental anatomic structures and the maternal and fetal circulations using Ferumoxytol and image post-processing, such as using volume rendering techniques described above. Depiction of this anatomy in vivo is heretofore unprecedented and, thus, the provided systems and method enable new insights into placental function during pregnancy and allow possible therapeutic interventions. That is the systems and methods provided herein provide the opportunity to make pathologic diagnoses during pregnancy, which were previously possible only after birth. This knowledge enables clinicians to tailor therapeutic interventions during gestation.

The invention has been described according to one or more preferred embodiments, and it should be appreciated that many equivalents, alternatives, variations, and modifications, aside from those expressly stated, are possible and within the scope of the invention. The preceding discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

It is to be understood that the disclosure is not limited in its application to the details of construction and the arrangement of components set forth in the description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

As used in the claims, the phrase “at least one of A, B, and C” means at least one of A, at least one of B, and/or at least one of C, or any one of A, B, or C or combination of A, B, or C. A, B, and C are elements of a list, and A, B, and C may be anything contained in the Specification.

We claim:

1. A magnetic resonance (MR) imaging system comprising:
a magnet system configured to generate a static magnetic field (Bo) about at least a portion of a subject including an organ receiving blood from multiple, different supplies and having received a dose of a material configured to change MR contrast;
a plurality of gradient coils configured to apply magnetic gradients to the static magnetic field;
a radio frequency (RF) system configured to apply an excitation field to the subject and acquire MR image data from the subject;
a computer system programmed to:
acquire an MR dataset from the organ receiving the blood from multiple, different supplies;
reconstruct the MR dataset into a first set of MR images using a first reconstruction process configured to weight the first set of MR images to the material configured to change the MR contrast;
reconstruct the MR dataset into a second set of MR images using a second reconstruction process configured to weight the second set of MR images against the material configured to change the MR contrast; and
a display configured to display the first set of MR images and the second set of MR images together with a user-selectable weighting of the first set of MR images relative the second set of MR images to independently view the multiple, different supplies of blood to the organ.

2. The system of claim 1, wherein the first set of MR images includes maximum intensity projection images and the second set of MR images includes minimum intensity projection images.

3. The system of claim 1, wherein the computer system is configured to acquire the MR dataset from the organ receiving the blood from multiple, different supplies during a single MR acquisition.

4. The system of claim 1, wherein the organ includes a placenta and the multiple, different supplies include a supply from a mother and a supply from a fetus.

5. The system of claim 1, wherein the material configured to change MR contrast includes iron.

6. The system of claim 1, wherein the material configured to change MR contrast includes Ferumoxytol.

7. A method for imaging a portion of a subject including an organ receiving blood from multiple, different supplies using a magnetic resonance imaging (MRI) system, the method comprising:
delivering a dose of a material configured to change MR contrast to the subject;
following delivery of the dose of the material configured to change MR contrast to the subject, simultaneously acquiring MR data from multiple, different supplies to form an MR dataset;
reconstructing the MR dataset into a first set of MR images using a first reconstruction process configured to weight the first set of MR images to the material configured to change the MR contrast;
reconstructing the MR dataset into a second set of MR images using a second reconstruction process configured to weight the second set of MR images against the material configured to change the MR contrast;
receiving a user-selectable weighting of the first set of MR images relative the second set of MR images to independently view the multiple, different supplies of blood to the organ; and
displaying the first set of MR images and the second set of MR images together based on the user-selectable weighting of the first set of MR images relative the second set of MR images to independently view the multiple, different supplies of blood to the organ.

8. The method of claim 7, wherein the first set of MR images includes maximum intensity projection images and the second set of MR images includes minimum intensity projection images.

9. The method of claim 7, wherein the organ includes a placenta and the multiple, different supplies include a supply from a mother and a supply from a fetus.

10. The method of claim 7, wherein the material configured to change MR contrast includes iron.

11. The method of claim 8, wherein the material configured to change MR contrast includes Ferumoxytol.

12. A computer system including a non-transitory computer-readable medium having stored thereon instructions that, when executed by the computer, cause the computer to carry out steps comprising:
accessing MR data acquired from a region of interest (ROI) including a placenta of a subject having received a dose of a material configured to change MR contrast;
reconstructing the MR data into a set of maximum intensity projection (MIP) images;
reconstructing the MR data into a set of minimum intensity projection (MinIP) images;
receiving a user selection of one of a transparency or a weighting of the MIP images relative to the MinIP images; and
displaying the MIP images and the MinIP images as a composite image of the placenta formed based on the user selection.

13. A method for creating images of a placenta of a subject pregnant with a fetus, the method comprising:
accessing MR data acquired from a region of interest (ROI) including a placenta of a subject having received a dose of a material configured to change MR contrast;
reconstructing the MR dataset into a first set of MR images using a first reconstruction process configured to weight the first set of MR images to placental blood flow of the subject;
reconstructing the MR dataset into a second set of MR images using a second reconstruction process configured to weight the second set of MR images to placental blood flow of the fetus; and
displaying the first set of MR images and the second set of MR images together to selectively display placental blood flow of the subject or placental blood flow of the fetus.

14. The method of claim 13, further comprising receiving a user selection of a weighting between placental blood flow of the subject or placental blood flow of the fetus.

15. The method of claim 14, wherein the step of displaying includes forming a composite image from the first set of MR images and the second set of MR images based on the user selectin of the weighting.

16. The method of claim 14, further comprising displaying a user interface to receive the user selection and receive adjustments of the user selection.

17. The method of claim 13, wherein the first set of MR images includes maximum intensity projection images and the second set of MR images includes minimum intensity projection images.

18. A method for generating images of a portion of a subject including an organ receiving blood from multiple, different supplies, the method comprising:
accessing MR data acquired from a subject having received a dose of a material configured to change MR contrast, wherein the MR data was acquired from a region of interest (ROI) including an organ with blood flow from multiple, different supplies;
reconstructing the MR dataset into a first set of MR images using a first reconstruction process configured to weight the first set of MR images to the material configured to change the MR contrast;
reconstructing the MR dataset into a second set of MR images using a second reconstruction process configured to weight the second set of MR images against the material configured to change the MR contrast; and
displaying the first set of MR images and the second set of MR images to selectively display blood flow from each of the multiple, different supplies.

19. The method of claim 18, wherein the organ is a placenta.

20. The method of claim 18, wherein the first set of MR images includes maximum intensity projection images and the second set of MR images includes minimum intensity projection images.
